(19) United States
(12) Patent Application Publication
(10) Pub. No.: US 2024/0350816 A1
(43) Pub. Date: Oct. 24, 2024

(54) DEVICES WITH INTEGRATED CONCAVE COILS

(71) Applicant: Cochlear Limited, Macquarie University, NSW (AU)

(72) Inventors: Harald KREICERS, Marsfield, NSW (AU); Marcus SWIFT, Epping, NSW (AU); Aleesha CALDWELL, Woy woy, NSW (AU); Nathan ISAACSON, Balgowlah, NSW (AU); William WIDJAJA, North Ryde, NSW (AU)

(21) Appl. No.: 18/682,343

(22) PCT Filed: Aug. 4, 2022
(86) PCT No.: PCT/IB2022/057261

§ 371 Date: Feb. 8, 2024

Related U.S. Application Data
(60) Provisional application No. 63/233,381, filed on Aug. 16, 2021.

(51) Int. Cl. A61N 1/378; A61N 1/36; H01F 27/28; H02J 50/10; H04R 25/00

(52) U.S. Cl. CPC A61N 1/3787; A61N 1/36038; A61N 1/36046; H01F 27/2804; H01F 27/2823; H02J 50/10; H04R 25/554; H04R 25/609; H04R 25/65; H04R 2225/021; H04R 2225/67

(57) ABSTRACT

Devices having an inductive coil configured to be positioned proximate to an inductive coil in a secondary device (e.g., an implantable component) for the transfer of signals (e.g., power signals and/or data signals) there between. One or more of the inductive coils includes at least one concave portion formed into the shape/perimeter thereof.

### BACKGROUND

### Field of the Invention

The present invention relates generally to devices comprising integrated coils having at least one concave portion formed into the perimeter thereof.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a behind-the-ear device is provided. The behind-the-ear device comprises: a housing configured to be worn on an ear of a recipient; and an inductive coil located within the housing, wherein the inductive coil includes at least one concave portion formed in a perimeter of the inductive coil.

In another aspect, an inductive coil is provided. The inductive coil comprises: a semi-circular base; a semi-circular apex; a first side connecting a first end of the semi-circular base to a first end of the semi-circular apex; and a second side connecting a second end of the semi-circular base to a second end of the semi-circular apex, wherein at least the first side comprises a continuous concave curve.

In another aspect, an apparatus is provided. The apparatus comprises: a housing having a concave curved first edge and a convex curved second edge; and an inductive coil located within the housing, wherein the inductive coil has an elongate length with a first elongate side positioned adjacent the concave curved first edge of the housing and a second elongate side positioned adjacent the convex curved second edge of housing, and wherein the first elongate side of the inductive coil has a concave curvature.

In another aspect, an implantable medical device system is provided. The implantable medical device system comprises: an implantable component comprising an implantable coil; and an external component, comprising: a housing configured to be worn on an ear of a recipient, a concave external coil located within the housing, and a transceiver electrically connected to the concave external coil, wherein the transceiver is configured to transcutaneously transfer signals to the implantable coil via the concave external coil.

In another aspect, a method is provided. The method comprises: positioning a behind-the-ear device on an ear of a recipient, wherein the behind-the-ear device comprises a housing and an external concave coil located within the housing, and wherein an implantable component comprising an implantable coil configured to be implanted in the recipient; and transcutaneously transferring signals between the behind-the-ear device and the implantable component via the external concave coil and the implantable coil.

In another aspect, a behind-the-ear device is provided. The behind-the-ear device comprises: a housing having a concave curved first edge and a convex curved second edge; an reniform-shaped inductive coil located within the housing, and an electrical connector configured to electrically connect the inductive coil to a transceiver, wherein the inductive coil has an elongate length with a first elongate side positioned adjacent the concave curved first edge of the housing and a second elongate side positioned adjacent the convex curved second edge of housing, and wherein the first elongate side of the inductive coil has a concave curvature, and the second elongate side of the inductive coil has a convex curvature, and wherein the first elongate side and the second elongate side of the inductive coil each have length of at least forty (40) degrees. wherein the inductive coil comprises an electrical connector configured to electrically connect the inductive coil to a transceiver.

### BRIEF DESCRIPTION OF THE DRAWINGS

Embodiments of the present invention are described herein in conjunction with the accompanying drawings, in which:

FIGS. 1A and 1B illustrate an example implantable medical device system, in accordance with certain embodiments presented herein;

FIG. 2A is a perspective view of a concave coil, in accordance with certain embodiments presented herein;

FIG. 2B is a top view of the concave coil of FIG. 2A;

FIG. 3 is a diagram illustrating polar (radius and angle) definitions for the concave coil of FIGS. 2A and 2B, in accordance with certain embodiments presented herein;

FIG. 4A is a perspective view of a concave coil, in accordance with certain embodiments presented herein;

FIG. 4B is a top view of the concave coil of FIG. 4A;

FIG. 5 illustrates a perspective view of the concave coil of FIGS. 2A and 2B aligned with the concave coil of FIGS. 4A and 4B;

FIG. 6 illustrates a top view of the concave coil of FIGS. 2A and 2B aligned with the concave coil of FIGS. 4A and 4B;

FIG. 7 illustrates an angular offset between the concave coil of FIGS. 2A and 2B and the concave coil of FIGS. 4A and 4B;

FIG. 8 illustrates a radial offset between the concave coil of FIGS. 2A and 2B and the concave coil of FIGS. 4A and 4B;

FIGS. 9A, 9B, 9C, 9D, 9E, and 9F are top views of different PCB concave coils, in accordance with certain embodiments presented herein;

FIG. 10 is a schematic diagram illustrating a concave coil within a BTE device, in accordance with certain embodiments presented herein;

FIG. 11 illustrates a functional block diagram of an implantable stimulator system that can benefit from the technologies described herein;

FIG. 12 illustrates a cochlear implant system that can benefit from use of the technologies disclosed herein;

FIG. 13 illustrates a retinal prosthesis system that comprises an external device, a retinal prosthesis, and a mobile computing device; and

FIG. 14 is a flowchart of an example method, in accordance with certain embodiments presented herein.

### DETAILED DESCRIPTION

Presented herein are devices having an inductive coil configured to be positioned proximate to an inductive coil in a secondary device (e.g., an implantable component) for the transfer of signals (e.g., power signals and/or data signals) there between. One or more of the inductive coils includes at least one concave portion formed into the shape/perimeter thereof. An inductive coil having at least one concave portion formed into the shape/perimeter thereof is sometimes referred to herein as a “concave coil” (e.g., sections of concavity in the coil's winding contour). In contrast, the term “non-concave coil” is sometimes used herein to refer to an inductive coil that do not include a concave portion formed into the shape/perimeter thereof.

In certain embodiments, the technologies described herein include positioning a wearable device (e.g., a behind-the-ear sound processor) having an external concave coil therein proximate to an implantable component (e.g., of a cochlear implant) having a concave or non-concave implantable coil. While magnets are traditionally used to align external and implantable coils, not all implantable components have retention magnets (e.g., for reasons of MRI compatibility) usable for such purposes. Further, magnets in external devices can add bulk. The techniques disclosed herein can be used to facilitate effective and efficient wireless transfer of signals (e.g., wireless communication) between an external coil and an implantable coil without the need for magnets. That is, in accordance with embodiments presented herein, a wearable device can be positioned relative to an implantable component such that a concave external coil in the wearable device is able to communicate with an implantable coil in the implantable component, even if the external concave coil and implantable coil are not directly aligned with one another (e.g., an angular offset is present). Thus, implementations can provide an alternative to traditional magnet-located coil implementations and be able to communicate with magnet-less implantable devices.

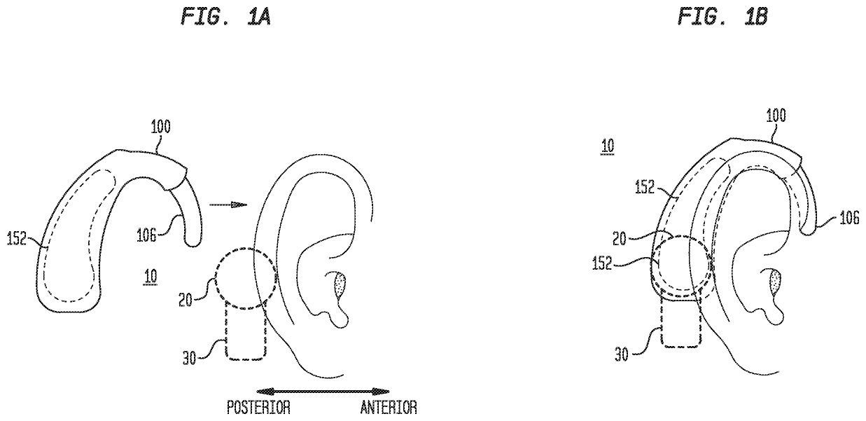

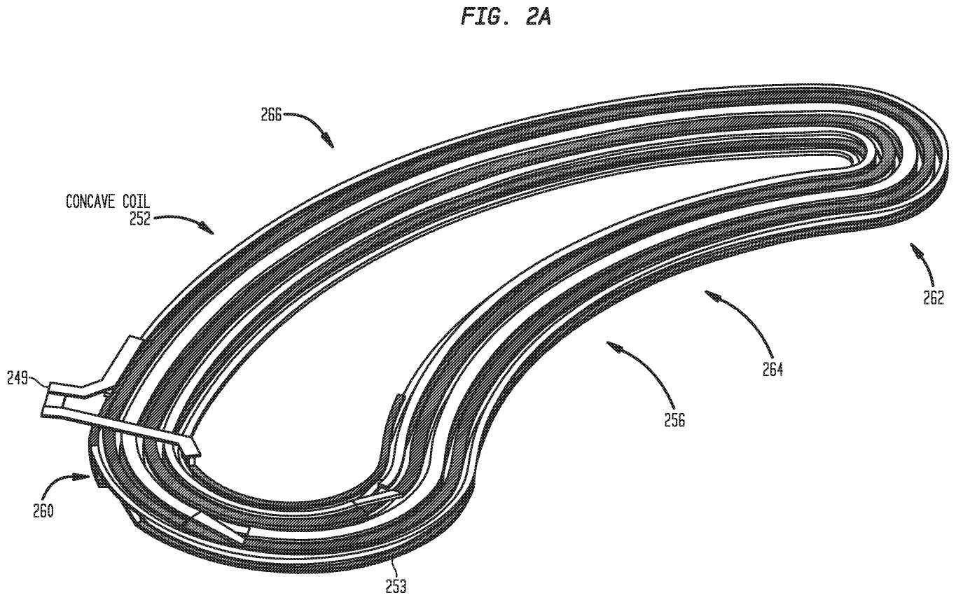

FIGS. 1A and 1B illustrate an example implantable medical device system 100 that includes an implantable component 30 and a wearable device 100. In this example, the wearable device 100 includes a concave external coil 152 and the implantable component 30 includes a non-concave implantable coil 20.

FIG. 1A shows the wearable device 100 being placed on a recipient's ear with respect to the implantable coil 20 of the implantable component 30 within the recipient, while FIG. 1B shows the wearable device 100 worn on the recipient's ear, with the concave coil 152 being disposed proximate the implantable coil 20. As shown, in this example, the wearable device 100 is configured to be worn with respect to a recipient's ear and can be referred to as an ear-worn device and, more specifically, a behind-the-ear (BTE) device.

In an example, the implantable device 30 is an implantable medical device, such as a cochlear implant, a vestibular implant, or a dedicated tinnitus therapy device, among other devices. In such examples, the implantable device 30 can be configured to be implanted proximate a mastoid cavity of a recipient. However, it is to be appreciated that the techniques presented herein may also be partially or fully implemented by other types of implantable medical devices. For example, the techniques presented herein may be implemented by other auditory prosthesis systems that include one or more other types of auditory prostheses, such as middle ear auditory prostheses, bone conduction devices, direct acoustic stimulators, electro-acoustic prostheses, auditory brain stimulators, combinations or variations thereof, etc. In further embodiments, the presented herein may also be implemented by, or used in conjunction with, visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

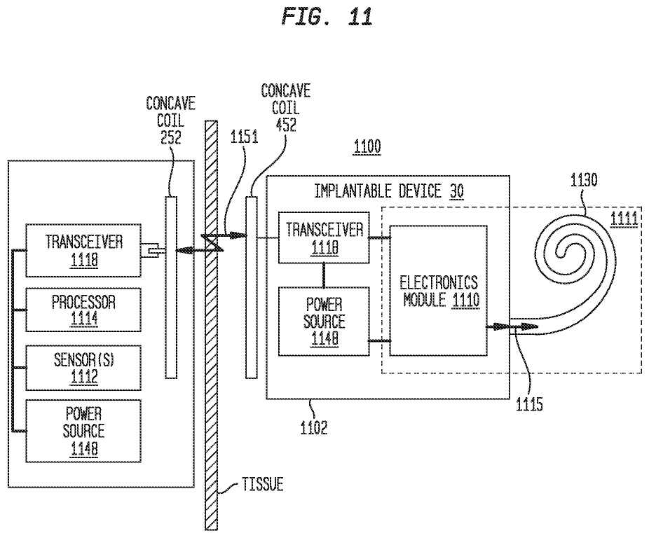

Returning to the examples of FIGS. 1A and 1B, the implantable coil 20 can be a component that is configured to receive or transmit a signal, such as via an inductive arrangement formed by multiple turns of wire. As noted, the wearable device 100 is a device configured to be worn by a recipient and is configured to communicate, using the concave coil 152, with the implantable coil 20. The wearable device 100 can be configured for any of a variety of functions, such as external sensing, charging, or processing for the implantable component 30. The wearable device 100 can take any of a variety of forms. In the illustrated example, the wearable device 100 is arranged as a behind-the-ear device, such as for a hearing aid or an auditory prosthesis. Example components and features of a wearable device 100 are described in relation to FIGS. 11-13, below.

As illustrated, the wearable device 100 includes a retainer 106. The retainer 106 is a component or portion of the wearable device 100 configured to permit the wearable device 100 to be wearably retained by the recipient. In the illustrated example, the retainer 106 is in the form of an ear hook. The ear hook can be a curved component or portion of housing extending from a main body of the wearable device 100. The curve of the ear hook 106 is configured to rest along a portion of the top of the recipient's pinna adjacent the side of the recipient's head. When the ear hook 106 rests along a portion of the top of the recipient's pinna adjacent the side of the recipient's head, the wearable device 100 can be hung on the recipient's ear, thereby wearably retaining the wearable device 100 on the recipient's ear. In an example, the retainer 106 is a clip configured to clip onto a portion of the recipient's body, clothing, or hair. In an example, the retainer 106 is a component configured to be inserted in the recipient's ear (e.g., into the pinna or the ear canal) and retain the device by a fit therein. In an example, the wearable device 100 is arranged as a pair of eyeglasses, with the retainer 106 being the temples (e.g., a curved portion of the temples).

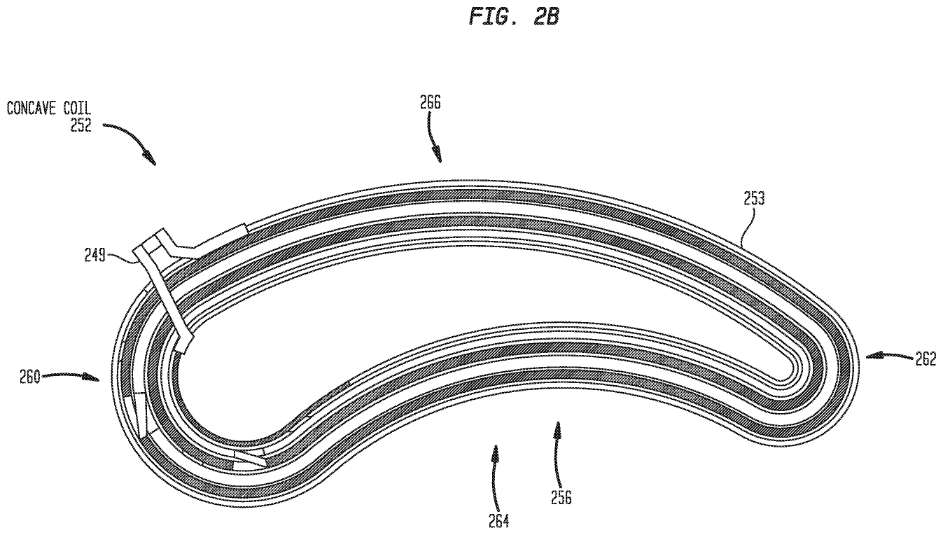

The concave external coil 152 can be a component configured to receive or transmit a signal, such as via an inductive arrangement formed by multiple turns of wire, a printed circuit board (PCB), etc. For instance, in certain embodiments the concave external coil 152 can take the form of a wound wire coil (e.g., wound copper, aluminum, platinum, etc.), while in other embodiments the concave external coil 152 includes a substrate to which the turns of wire are affixed or in which the turns of wire are disposed. As described further below, a concave external coil, such as coil 152, includes at least one concave portion formed into the shape/perimeter thereof. However, in addition, a concave external coil presented can have a number of different physical dimensions, arrangements, etc. FIGS. 2A, 2B, 3, 4A, 4B, and 9A-9F illustrate further details of example concave coils, in accordance with embodiments presented herein.

FIG. 2A is a perspective view of a first concave coil 252, in accordance with embodiments presented herein, while FIG. 2B is top view of the first concave coil 252. In this specific example, the first concave coil 252 is formed by a plurality of conductive tracks 253 on a printed circuit board (PCB) substrate. Coils formed on a PCB substrate, such as first concave coil 252, are sometimes referred to as PCB coils. For ease of illustration, FIGS. 2A and 2B illustrate only the conductive tracks 253 (e.g., the PCB substrate has been omitted to show just the conductive tracks). It is to be appreciated that the PCB coil example of FIGS. 2A and 2B is merely illustrative and that the first concave coil 252 could alternatively be formed in other manners, such as a wire-wound coil. In operation, the first concave coil 252 is electrically connected to one or more other electrical components via an electrical connector 249.

As described elsewhere herein, a concave coil, such as first concave coil 252, is an inductive coil with at least one concave portion formed into the shape/perimeter thereof (e.g., a concavity formed into a section of the coil winding contour). As used herein, the terms “concave” and “convex” are defined relative to a center of the corresponding coil. That is, a concave portion/region extends inwards towards the center of the coil, while a convex portion/region extends outwards from the center of the corresponding coil/

In FIGS. 2A and 2B, the concave portion is represented by arrow 256. In general, the concave portion 256 is an inward curve formed in each of the plurality conductive tracks 253 in the same location. The first concave coil 252 is sometimes referred to herein as having a “reniform” shape or, alternatively, as a “reniform-shaped” coil.

The first concave coil 252 is generally described as comprising a base portion 260, an apex portion 262, a first side 264, and a second side 266. In this example, the concave portion 256 is located at the first side 264 of the first concave coil 252. It is to be appreciated that concave portion 256 could alternatively be located at other portions of the first concave coil 252, such as at second side 266. It is also to be appreciated that a concave coil could include multiple concave portions (e.g., concave portions at each of the first side 264 and the second side 266).

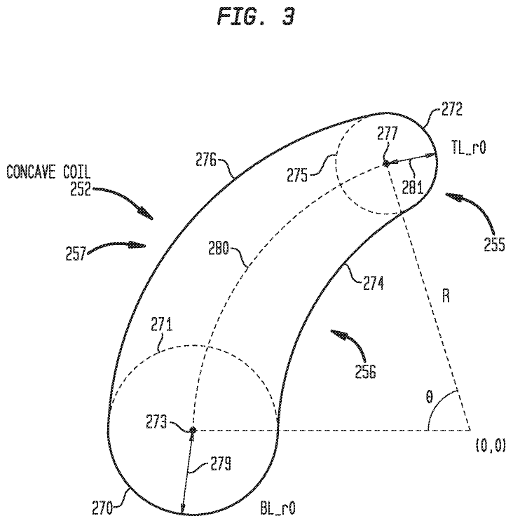

FIG. 3 is a simplified line drawing of the first concave coil 252 illustrating further details regarding the physical configuration of the coil. In FIG. 3, line 255 represents the outer perimeter/shape of the first concave coil 252, as formed by the plurality of conductive tracks 253. The first concave coil 252 can generally be described as including a base arc 270 corresponding to base portion 260 (FIGS. 2A and 2B), an apex arc 272 corresponding to apex portion 272 (FIGS. 2A and 2B), a first side arc 274 corresponding to first side 264 (FIGS. 2A and 2B), and a second side arc 276 corresponding to second side 266. The first side arc 274 and the second side arc 276 are each connected to opposing ends of the base arc 270 and the apex arc 272.

In the illustrative example of FIG. 3, the base arc 270 forms part of a base circle 271 having a center point 273, while the apex arc 272 forms part of an apex circle 275 having a center point 277. In the example of FIG. 3, the first side arc 274 includes the concave portion 256. More specifically, the first side arc 274 (e.g., the portions of the conductive traces 253 at a first side 264 of the coil 252) is formed as an inward curve that forms/defines the concave portion 256. Conversely, in the example of FIG. 3, the second side arc 276 (e.g., the portions of the conductive traces 253 at a second side 266 of the coil 252) is formed as an outward curve that forms/defines a convex portion 257. Stated differently, the first side arc 274 has a concave shape and the second side arc 276 has a convex shape.

As shown, the base arc 270 has a radius 279 that is generally defined as the distance between the center point 273 of the base circle 271 and the portion of the perimeter 255 forming the base arc 270. Similarly, the apex arc 272 has a radius 281 that is generally defined as the distance between the center point 277 of the base circle 275 and the portion of the perimeter 255 forming the apex arc 272. In the example of FIGS. 2A, 2B, and 3, the radius 281 of the apex arc 272 is less than the radius 279 of the base arc 270. That is, in the example of FIGS. 2A, 2B, and 3, the base arc 270 is longer than the apex arc 272.

The first concave coil 252 also has a center arc 280 with a length 281, which is generally defined as the distance between center point 273 of the base circle 271 and center point 277 of the apex circle 275. In certain embodiments, the length 281 of the center arc 280 is at least two (2) times the radius 279 of the base arc 270 (e.g., at least twice the base arc radius). In certain embodiments, the first side arc 274 and the second side arc 276 each have an angular length (0) of at least forty (40) degrees, where the angular length is defined by the angle between center point 273 of the base circle 271 and center point 277 of the apex circle 275. The side arc lengths generally correspond to the center arc length 280.

FIGS. 2A, 2B, and 3 generally illustrate one example physical configuration for a concave coil in accordance with embodiments presented herein. As described elsewhere herein, concave coils presented herein can have a number of different shapes and physical configurations that are different than those shown in FIGS. 2A, 2B, and 3.

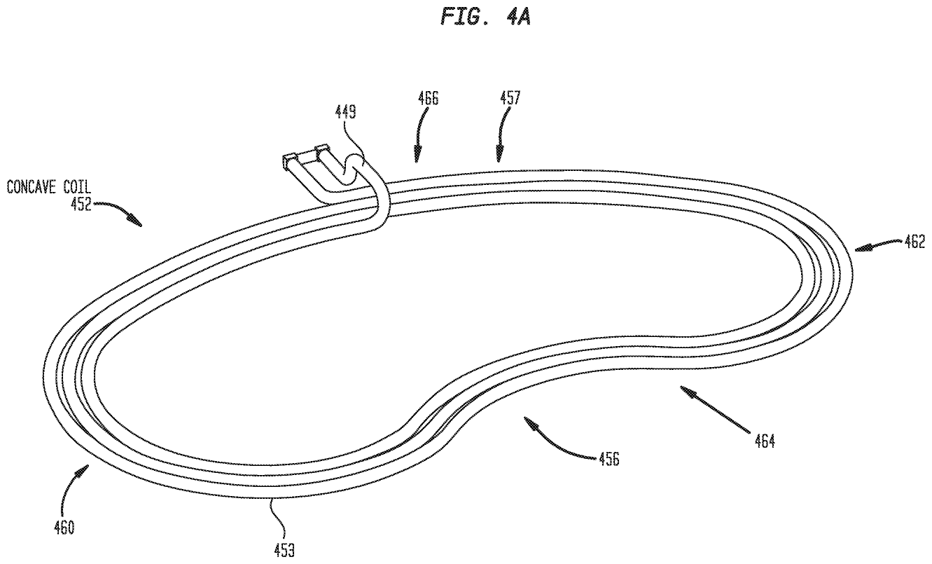

For example, FIG. 4A is a perspective view of a second concave coil 452, in accordance with embodiments presented herein, while FIG. 4B is top view of the second concave coil 452. In this specific example, the second concave coil 452 is a wire-wound coil formed by a plurality of wire loops 453. It is to be appreciated that the wire-wound coil example of FIGS. 4A and 4B is merely illustrative and that the second concave coil 452 could alternatively be formed in other manners, such as a PCB coil. In operation, the second concave coil 452 is electrically connected to one or more other electrical components via an electrical connector 449.

As described elsewhere herein, a concave coil, such as second concave coil 452, is an inductive coil with at least one concave portion formed into the shape/perimeter thereof. In FIGS. 4A and 4B, the concave portion is represented by arrow 456. In general, the concave portion 456 is an inward curve formed in each of the plurality conductive tracks 453 in the same location. Similar to the above, the second concave coil 452 is sometimes referred to herein as having a reniform shape.

The second concave coil 452 is generally described as comprising a base portion 460, an apex portion 462, a first side 464, and a second side 466. In this example, the concave portion 456 is located at the first side 464 of the second concave coil 452. It is to be appreciated that concave portion 456 could alternatively be located at other portions of the second concave coil 452, such as at second side 466. It is also to be appreciated that a concave coil could include multiple concave portions (e.g., concave portions at each of the first side 464 and the second side 466).

In the examples of FIGS. 4A and 4B, the second side 466 (e.g., the portions of the wire looks 453 at the second side 466 of the coil 452) is formed as an outward curve that forms/defines a convex portion 457. Stated differently, the first side 464 of the second concave coil 452 has a concave shape and the second side 466 of the second concave coil 452 has a convex shape.

In the examples of FIGS. 4A and 4B, the definitions (e.g., polar and angular definitions) described with reference to the first concave coil 252 in FIG. 3 are also applicable to the second concave coil 452. That is, the second concave coil 452 also comprises an outer perimeter/shape, as formed by the plurality of wire loops 453, including a base arc corresponding to base portion 460, an apex arc corresponding to apex portion 472, a first side arc corresponding to first side 464, and a second side arc corresponding to second side 266. For ease of description, the description of FIG. 3 is not repeated with specific reference to second concave coil 452.

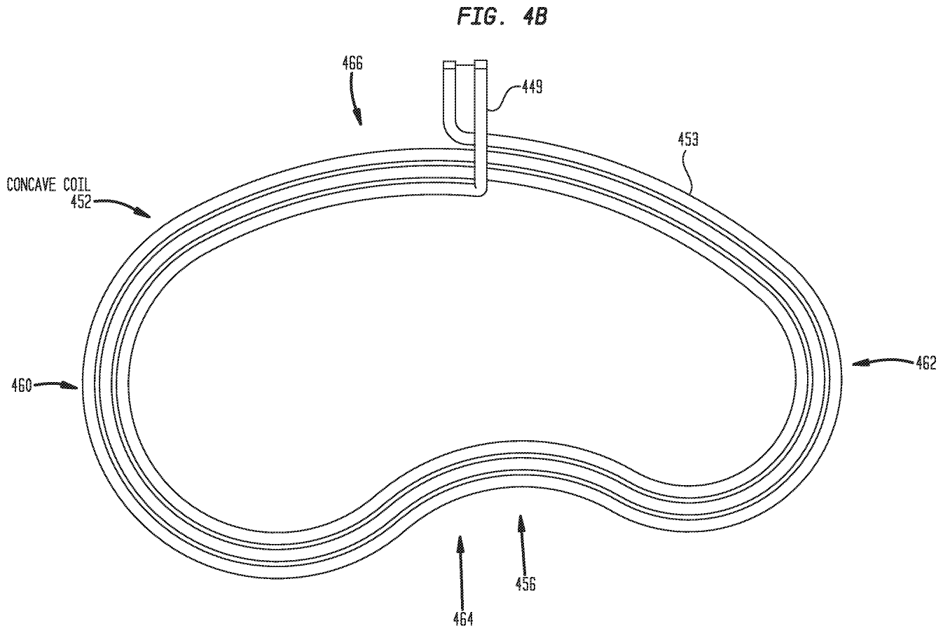

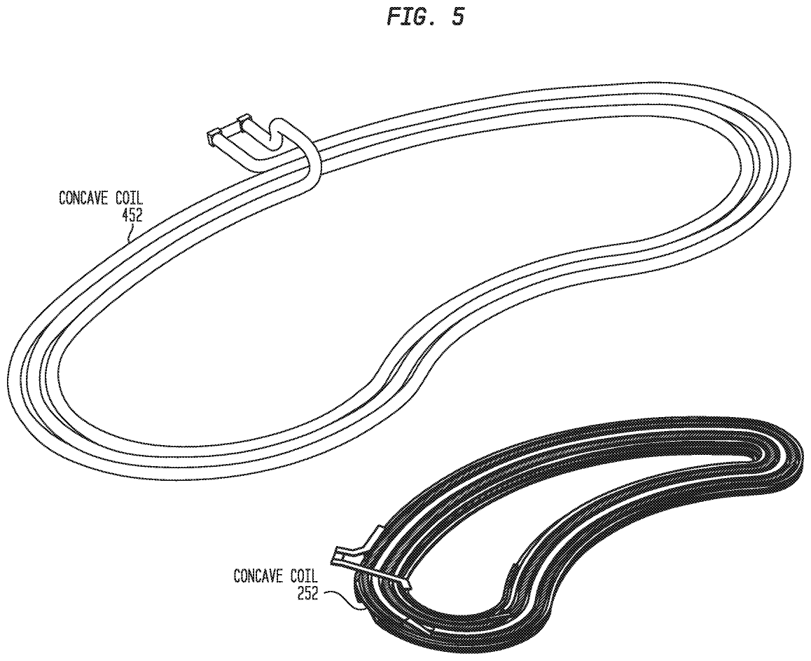

As noted, in certain embodiments, concave inductive coils, in accordance with embodiments presented herein, can advantageously be used for the transcutaneous transfer of signals (e.g., power and/or data signals) between an external device and an implantable medical device. FIGS. 5 and 6 are schematic diagrams illustrating use of first concave coil 252 (FIGS. 2A and 2B) and second concave coil 452 for the transcutaneous transfer of signals.

In the examples of FIGS. 5 and 6, the first concave coil 252 is an “external” coil, meaning it is located outside of the body of the recipient. As such, in these examples, the first concave coil 252 can be referred to as “external concave coil 252.” In certain embodiments, the external concave coil 252 can be located in a BTE device. Conversely, the second concave coil 452 is an “implantable” coil, meaning it is located inside of the body of the recipient. As such, in these examples, the second concave coil 452 can be referred to as “implantable concave coil 452.”

FIG. 5 is a perspective view of an arrangement in which the first concave coil 252 is aligned with the second concave coil 452, while FIG. 6 is a bottom view the first concave coil 252 aligned with the second concave coil 452. As shown in FIG. 6, the first concave coil 252 and the second concave coil 452 are “aligned” when the center point of the base arc of coil 252 and the center point of the apex arc of coil 252 are substantially co-axial with the center point of the base arc of coil 452 and the center point of the apex arc of coil 452, respectively . . . Alternatively, the first concave coil 252 and the second concave coil 452 are “aligned” when the distance between the windings of the two coils is equal about the whole perimeter.

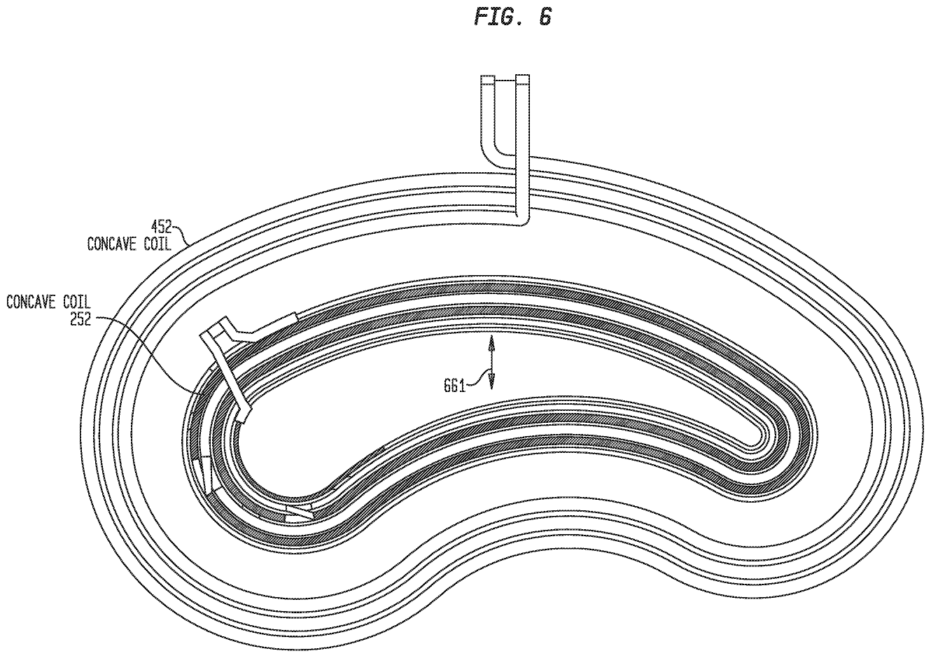

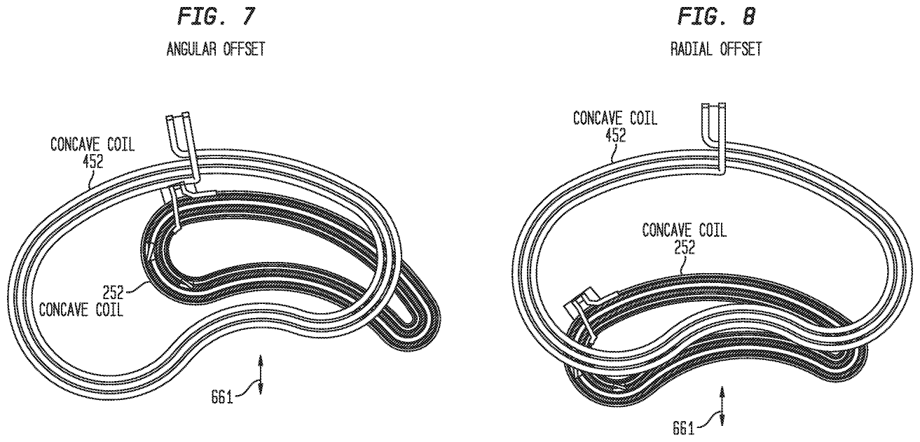

FIG. 7 is a bottom view of an arrangement in which there is an angular/offset between the first concave coil 252 and the second concave coil 452. That is, in FIG. 7, the first concave coil 252 is rotated angularly relative to the second concave coil 452 (e.g., angular offset corresponds to relative angular rotation of one of the coils relative to the other of the coils) . . . . FIG. 8 is a bottom view of an arrangement in which there is a radial offset between the first concave coil 252 and the second concave coil 452, meaning there is a lateral shift between the center arcs of the first concave coil 252 and the second concave coil along an axis 661.

In generally, two coils most efficiently transfer signals when the coils are aligned with one another, as shown in FIGS. 5 and 6. Moreover, conventional non-concave (e.g., circular) coils are less efficient, or even unable to transfer signals, in the presence of angular or radial offsets (e.g., conventional transcutaneous transfer systems are sensitive to both radial and angular offsets). In contrast, transcutaneous transfer systems including one or more concave coils are still able to efficiently operate in the presence of angular offsets, as shown in FIG. 7. Stated differently, the use of one or more concave coils increases coil coupling factor in a lateral degree of freedom. The increased coupling factor in a lateral degree of freedom (e.g., with an angular offset) is due to the fact that for a given angular offset, a larger percentage of the coils will remain overlapping when compared with a set of circular coils of the same total area.

In addition to being less sensitive to angular offsets, the concave coils presented herein provide a number of other advantages over convention non-concave coils. In particular, the concave coils presented herein can provide increased efficiency in a wireless power transfer systems where space and geometry of the coils is restricted. For example, in a wireless power transfer link where the coil geometry is restricted (such as the form-factor of a BTE processor), the concave coils are curved with the shape of the BTE housing in order to maximize the available coil area. That is, the concave coil allows the coil area to be maximized when confined to a restricted shape and increases the coupling factor between the transmitter and receiver coils when compared to the largest possible circular coil. As noted, the use of one or more concave coils also reduces the roll-off of the link coupling factor when the transmitter and receiver are misaligned along a central axis of the coils. That is, in accordance with embodiments presented herein, an external device (wearable device) having an external concave coil can be positioned relative to an implantable component such that the concave external coil is able to communicate with an implantable coil in the implantable component, even if the external concave coil and the implantable coil are not directly aligned with one another (e.g., an angular offset is present). Thus, implementations can provide an alternative to traditional magnet-located coil implementations and be able to communicate with magnet-less implantable devices. In such embodiments, the external device and the implantable component are sometimes referred to as “magnet-less” devices.

In view of the above, the use of one or more concave coils, such as shown in FIGS. 5-8, increases coupling factor of an inductive system in a restricted geometry and increased link efficiency, leading to improvements in battery life and device longevity. The use of one or more concave coils can also remove the need for a coil array and complex related circuitry, while reducing the coil performance roll-off associated with misalignment in specific degrees of freedom.

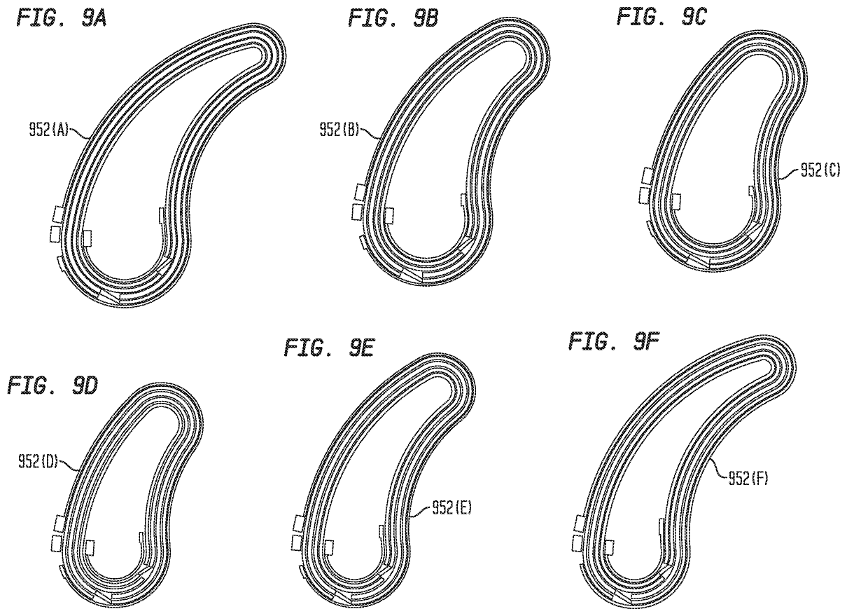

As noted, FIGS. 2A-2B, 3, 4A, and 4B illustrate two example concave coil arrangements, in accordance with embodiments presented herein. However, also as noted above, concave coils in accordance with embodiments presented herein can have a number of different physical configurations, including different sizes, shapes, etc. FIGS. 9A-9F illustrate six (6) specific physical configurations for concave coils in accordance with certain embodiments presented herein. It is to be appreciated that the specific physical configurations shown in FIGS. 9A-9F, and described further below, are merely illustrative and that the concave coils presented herein are not limited to any of these specific arrangements, unless otherwise noted herein.

Referring first to FIG. 9A, shown is a top view of concave coil 952 (A) having a 12 mm base arc (e.g., the diameter of the base arc is 12 mm), and a center arc length of 70 degrees. FIG. 9B is a top view of concave coil 952 (B) having a 12 mm base arc (e.g., the diameter of the base arc is 12 mm), and a center arc length of 55 degrees. FIG. 9C is a top view of concave coil 952 (C) having a 12 mm base arc (e.g., the diameter of the base arc is 12 mm), and a center arc length of 40 degrees. FIG. 9D is a top view of concave coil 952 (D) having a 10 mm base arc (e.g., the diameter of the base arc is 10 mm), and a center arc length of 40 degrees. FIG. 9E is a top view of concave coil 952 (E) having a 10 mm base arc (e.g., the diameter of the base arc is 10 mm), and a center arc length of 55 degrees. Finally, FIG. 9F is a top view of concave coil 952 (F) having a 10 mm base arc (e.g., the diameter of the base arc is 10 mm), and a center arc length of 70 degrees. Again, it is to be appreciated that the specific base arc diameters and center arc lengths shown in FIGS. 9A-9F are merely illustrative.

In FIGS. 9A-9F, the coils each include an apex arc where, for the 70° coils, the apex radius is 3 mm for both the 10 mm and 12 mm coils. For the shorter coils, the apex arc is increased linearly from 3 mm to the base arc radius as the angle is shortened. Therefore, in FIGS. 9A-9F, the apex arc radii are as follows:

$$6 mm - 3 mm * ( 70 / 70 ) = 3. mm \qquad FIG . 9 A$$

$$6 mm - 3 mm * ( 55 / 70 ) = 3.64 mm \qquad FIG . 9 B$$

$$6 mm - 3 mm * ( 40 / 70 ) = 4.29 mm \qquad FIG . 9 C$$

$$5 mm - 2 mm * ( 70 / 70 ) = 3. mm \qquad FIG . 9 D$$

$$5 mm - 2 mm * ( 55 / 70 ) = 3.43 mm \qquad FIG . 9 E$$

$$5 mm - 2 mm * ( 40 / 70 ) = 3.86 mm \qquad FIG . 9 F$$

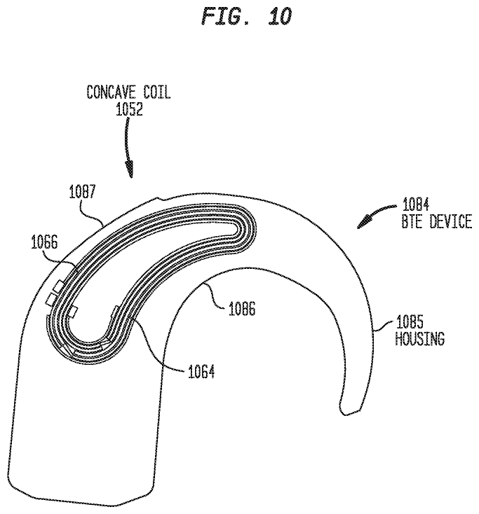

As noted above, concave coils in accordance with embodiments presented herein can advantageously be used in BTE devices. The concave coils presented herein are particularly suited for use in BTE devices because the concave has a shape/perimeter that substantially corresponds to that of the BTE device. This is generally shown in FIG. 10.

More specifically, shown in FIG. 10 is a simplified cross-sectional view of a BTE device 1084 comprising a housing 1085. As shown, the housing 1085 of the BTE device 1084 has a concave curved first side/edge 1086, and a convex curved second side/edge 1087. Shown in the housing 1085 is a concave coil 1052, in accordance with certain embodiments presented herein.

Similar to the housing 1085, the concave coil 1052 has a first concave side/edge 1064 that generally corresponds to the curvature of the concave curved first edge 1086 of the housing 1085. The concave coil 1052 also has a second convex side/edge 1066 and that generally corresponds to the curvature of the second convex edge 1087 of the housing 1085. That is, as shown, the first concave side/edge 1064 of the concave coil 1052 is positioned adjacent the concave curved first edge 1086 of the housing 1085, while the second convex side/edge 1066 is positioned the convex curved second edge 1086 of the housing 1085.

As noted above, the correspondence in curved sides between the BTE housing 1085 and the concave coil 1052 maximize the available coil area within the BTE housing, as compared to a conventional circular coil. That is, the concave coil 1052 is designed to maximize the coil area that is available within the BTE housing having opposing concave and convex surfaces.

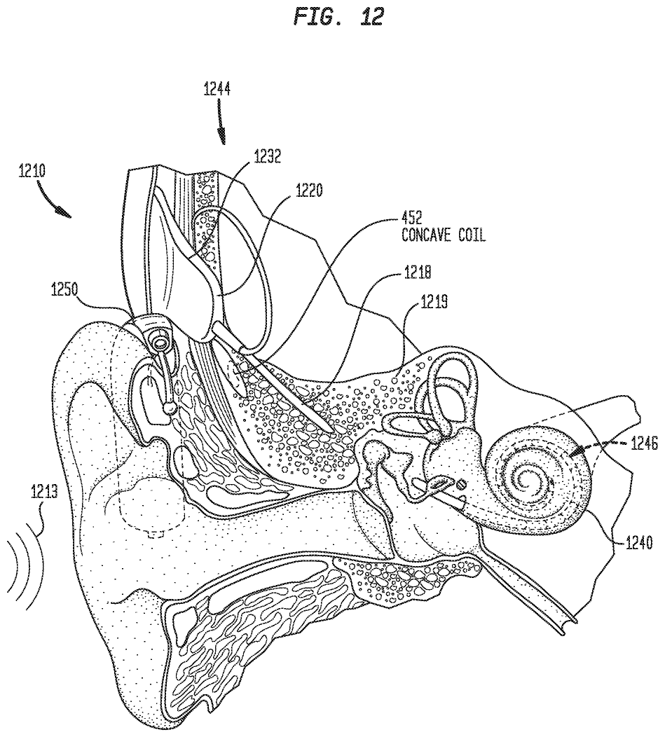

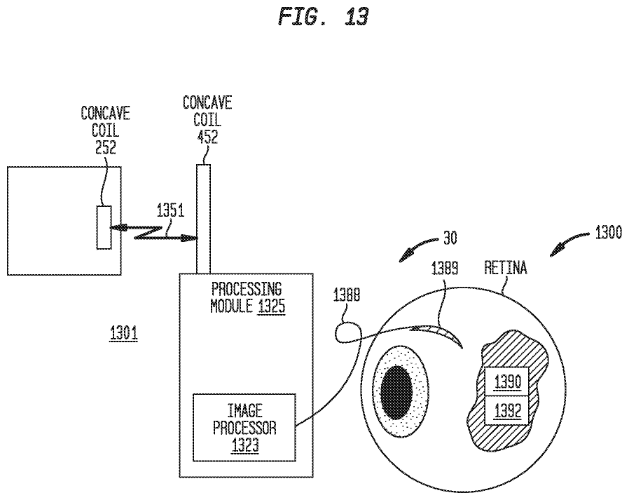

As previously described, the technology disclosed herein can be applied in any of a variety of circumstances and with a variety of different devices. Example devices that can benefit from technology disclosed herein are described in more detail in FIGS. 11-13, below. For example, the techniques described herein can be used with wearable medical devices, such as an implantable stimulation system as described in FIG. 11, a cochlear implant as described in FIG. 12, or a retinal prosthesis as described in FIG. 13. The technology can be applied to other medical devices, such as neurostimulators, cardiac pacemakers, cardiac defibrillators, sleep apnea management stimulators, seizure therapy stimulators, tinnitus management stimulators, and vestibular stimulation devices, as well as other medical devices that deliver stimulation to tissue. Further, technology described herein can also be applied to consumer devices. These different systems and devices can benefit from the technology described herein.

FIG. 11 is a functional block diagram of an implantable stimulator system 1100 that can benefit from the technologies described herein. The implantable stimulator system 1100 includes the wearable device 100 acting as an external processor device and an implantable device 30 acting as an implanted stimulator device. In examples, the implantable device 30 is an implantable stimulator device configured to be implanted beneath a recipient's tissue (e.g., skin). In examples, the implantable device 30 includes a biocompatible implantable housing 1102. Here, the wearable device 100 is configured to transcutaneously couple with the implantable device 30 via a wireless connection to provide additional functionality to the implantable device 30.

In the illustrated example, the wearable device 100 includes one or more sensors 1112, a processor 1114, a transceiver 1118, and a power source 1148. The one or more sensors 1112 can be one or more units configured to produce data based on sensed activities. In an example where the stimulation system 1100 is an auditory prosthesis system, the one or more sensors 1112 include sound input sensors, such as a microphone, an electrical input for an FM hearing system, other components for receiving sound input, or combinations thereof. Where the stimulation system 1100 is a visual prosthesis system, the one or more sensors 1112 can include one or more cameras or other visual sensors. Where the stimulation system 1100 is a cardiac stimulator, the one or more sensors 1112 can include cardiac monitors. The processor 1114 can be a component (e.g., a central processing unit) configured to control stimulation provided by the implantable device 30. The stimulation can be controlled based on data from the sensor 1112, a stimulation schedule, or other data. Where the stimulation system 1100 is an auditory prosthesis, the processor 1114 can be configured to convert sound signals received from the sensor(s) 1112 (e.g., acting as a sound input unit) into signals 1151. The transceiver 1118 is configured to send the signals 1151 in the form of power signals, data signals, combinations thereof (e.g., by interleaving the signals), or other signals. The transceiver 1118 can also be configured to receive power or data. Stimulation signals can be generated by the processor 1114 and transmitted, using the transceiver 1118, to the implantable device 30 for use in providing stimulation.

In the illustrated example, the implantable device 30 includes a transceiver 1118, a power source 1148, and a medical instrument 1111 that includes an electronics module 1110 and a stimulator assembly 1130. The implantable device 30 further includes a hermetically sealed, biocompatible implantable housing 1102 enclosing one or more of the components.

The electronics module 1110 can include one or more other components to provide medical device functionality. In many examples, the electronics module 1110 includes one or more components for receiving a signal and converting the signal into the stimulation signal 1115. The electronics module 1110 can further include a stimulator unit. The electronics module 1110 can generate or control delivery of the stimulation signals 1115 to the stimulator assembly 1130. In examples, the electronics module 1110 includes one or more processors (e.g., central processing units or microcontrollers) coupled to memory components (e.g., flash memory) storing instructions that when executed cause performance of an operation. In examples, the electronics module 1110 generates and monitors parameters associated with generating and delivering the stimulus (e.g., output voltage, output current, or line impedance). In examples, the electronics module 1110 generates a telemetry signal (e.g., a data signal) that includes telemetry data. The electronics module 1110 can send the telemetry signal to the wearable device 100 or store the telemetry signal in memory for later use or retrieval.

The stimulator assembly 1130 can be a component configured to provide stimulation to target tissue. In the illustrated example, the stimulator assembly 1130 is an electrode assembly that includes an array of electrode contacts disposed on a lead. The lead can be disposed proximate tissue to be stimulated. Where the system 1100 is a cochlear implant system, the stimulator assembly 1130 can be inserted into the recipient's cochlea. The stimulator assembly 1130 can be configured to deliver stimulation signals 1115 (e.g., electrical stimulation signals) generated by the electronics module 1110 to the cochlea to cause the recipient to experience a hearing percept. In other examples, the stimulator assembly 1130 is a vibratory actuator disposed inside or outside of a housing of the implantable device 30 and configured to generate vibrations. The vibratory actuator receives the stimulation signals 1115 and, based thereon, generates a mechanical output force in the form of vibrations. The actuator can deliver the vibrations to the skull of the recipient in a manner that produces motion or vibration of the recipient's skull, thereby causing a hearing percept by activating the hair cells in the recipient's cochlea via cochlea fluid motion.

The transceivers 1118 can be components configured to transcutaneously receive and/or transmit a signal 1151 (e.g., a power signal and/or a data signal). The transceiver 1118 can be a collection of one or more components that form part of a transcutaneous energy or data transfer system to transfer the signal 1151 between the wearable device 100 and the implantable device 30. Various types of signal transfer, such as electromagnetic, capacitive, and inductive transfer, can be used to usably receive or transmit the signal 1151. The transceiver 1118 can include or be electrically connected to a concave coil, such as concave coil 452 described above with reference to FIGS. 4A and 4B. As described elsewhere herein, in other embodiments the transceiver 1118 can include or be electrically connected to a non-concave coil instead of a concave coil.

As illustrated, the wearable device 100 includes a concave coil, such as concave coil 252 described above with reference to FIGS. 2A, 2B, and 3 for transcutaneous transfer of signals with the concave coil 452. As noted above, the transcutaneous transfer of signals between concave coil 252 and the concave coil 452 can include the transfer of power and/or data from the concave coil 252 to the concave coil 452 and/or the transfer of data from concave coil 452 to the concave coil 252. The power source 1148 can be one or more components configured to provide operational power to other components. The power source 1148 can be or include one or more rechargeable batteries. Power for the batteries can be received from a source and stored in the battery. The power can then be distributed to the other components as needed for operation.

As should be appreciated, while particular components are described in conjunction with FIG.11, technology disclosed herein can be applied in any of a variety of circumstances. The above discussion is not meant to suggest that the disclosed techniques are only suitable for implementation within systems akin to that illustrated in and described with respect to FIG. 11. In general, additional configurations can be used to practice the methods and systems herein and/or some aspects described can be excluded without departing from the methods and systems disclosed herein.

FIG. 12 illustrates an example cochlear implant system 1210 that can benefit from use of the technologies disclosed herein. The cochlear implant system 1210 includes an implantable component 1244 (which can correspond to implantable device 30) typically having an internal receiver/transceiver unit 1232, a stimulator unit 1220, and an elongate lead 1218. The internal receiver/transceiver unit 1232 permits the cochlear implant system 1210 to receive signals from and/or transmit signals to an external device 1250 (which can correspond to the wearable device 100). An external concave coil, such as concave coil 252 (not shown in FIG. 12), is located/disposed within the external device 1250.

The implantable component 1244 includes an implantable coil, such as concave coil 452, that, along with the internal receiver/transceiver unit 1232 and the stimulator unit 1220, are hermetically sealed within a biocompatible housing, sometimes collectively referred to as a stimulator/receiver unit. The elongate lead 1218 has a proximal end connected to the stimulator unit 1220, and a distal end 1246 implanted in a cochlea 1240 of the recipient. The elongate lead 1218 extends from stimulator unit 1220 to the cochlea 1240 through a mastoid bone 1219 of the recipient. The elongate lead 1218 is used to provide electrical stimulation to the cochlea 1240 based on the stimulation data. The stimulation data can be created based on the external sound 1213 using the sound processing components and based on sensory prosthesis settings. In certain examples, the external concave coil in the external device 1250 transmits electrical signals (e.g., power and/or stimulation data) to the concave coil 452 via a radio frequency (RF) link.

FIG. 13 illustrates a retinal prosthesis system 1301 that comprises an external device 1310 (which can correspond to the wearable device 100) and a retinal prosthesis 1300. The retinal prosthesis 1300 comprises an implanted processing module 1325 (e.g., which can correspond to the implantable device 30) and a retinal prosthesis sensor-stimulator 1390 is positioned proximate the retina of a recipient. The external device 1310 and the processing module 1325 can communicate via concave coils 252, 452. As described elsewhere herein, the concave coil 252 is located within a housing of the external device 1310, while concave coil 452 is configured to be implanted in the recipient. Signals 1351 can be transmitted/received (transferred) using the coils 252, 452.

In an example, sensory inputs (e.g., photons entering the eye) are absorbed by a microelectronic array of the sensor-stimulator 1390 that is hybridized to a glass piece 1392 including, for example, an embedded array of microwires. The glass can have a curved surface that conforms to the inner radius of the retina. The sensor-stimulator 1390 can include a microelectronic imaging device that can be made of thin silicon containing integrated circuitry that convert the incident photons to an electronic charge.

The processing module 1325 includes an image processor 1323 that is in signal communication with the sensor-stimulator 1390 via, for example, a lead 1388 which extends through surgical incision 1389 formed in the eye wall. In other examples, processing module 1325 is in wireless communication with the sensor-stimulator 1390. The image processor 1323 processes the input into the sensor-stimulator 1390, and provides control signals back to the sensor-stimulator 1390 so the device can provide an output to the optic nerve. That said, in an alternate example, the processing is executed by a component proximate to, or integrated with, the sensor-stimulator 1390. The electric charge resulting from the conversion of the incident photons is converted to a proportional amount of electronic current which is input to a nearby retinal cell layer. The cells fire and a signal is sent to the optic nerve, thus inducing a sight perception.

The processing module 1325 can be implanted in the recipient and function by communicating with the external device 1310, such as a behind-the-ear unit, a pair of eyeglasses, etc. The external device 1310 can include an external light/image capture device (e.g., located in/on a behind-the-ear device or a pair of glasses, etc.), while, as noted above, in some examples, the sensor-stimulator 1390 captures light/images, which sensor-stimulator is implanted in the recipient.

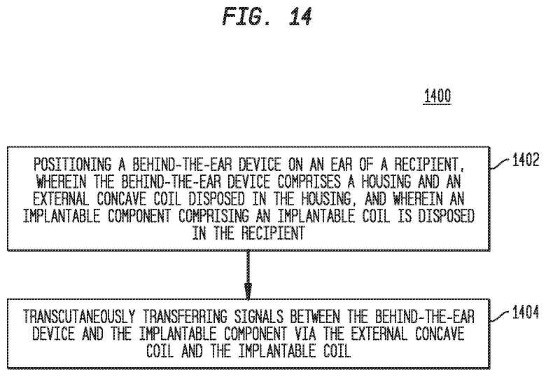

FIG. 14 is a flowchart illustrating an example method 1400, in accordance with certain embodiments presented herein. Method 1400 begins at 1402 where a behind-the-ear device is positioned on an ear of a recipient, wherein the behind-the-ear device comprises a housing and an external concave coil located within the housing, and wherein an implantable component comprising an implantable coil is configured to be implanted in the recipient. At 1404, signals are transcutaneously transferred between behind-the-ear device and the implantable component via the external concave coil and the implantable coil.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

1. A behind-the-ear device, comprising:
a housing configured to be worn on an ear of a recipient; and
an inductive coil located within the housing, wherein the inductive coil includes at least one concave portion formed in a perimeter of the inductive coil.

2. The behind-the-ear device of claim 1, wherein the inductive coil comprises a base arc, an apex arc, and first and second sides connecting the base arc and the apex arc, wherein at least one of the first and second sides includes the at least one concave portion.

3. The behind-the-ear device of claim 2, wherein the first and second sides comprise a first side arc and a second side arc, respectively, connecting opposing ends of the base arc and the apex arc.

4. The behind-the-ear device claim 3, wherein the first side arc has a concave shape and the second side arc has a convex shape.

5. The behind-the-ear device of claim 3, wherein the first side arc and the second side arc each have an arc length of at least forty (40) degrees.

6. The behind-the-ear device of claim 2, wherein the inductive coil has a center arc extending from a center of the base arc to a center of the apex arc, and wherein the center arc has a length that is at least two times a radius of the base arc.

7. (Currently Amended 1) The behind-the-ear device claim 2, wherein a radius of the apex arc is less than a radius of the base arc.

8. The behind-the-ear device of claim 2, wherein a radius of the apex arc is the same as a radius of the base arc.

9. The behind-the-ear device of claim 1, wherein the at least one concave portion comprises a plurality of concave portions formed in a perimeter of the inductive coil.

10. The behind-the-ear device of wherein claim 1, wherein the inductive coil is a printed circuit board (PCB) coil.

11. The behind-the-ear device of claim 1, wherein the inductive coil is a wire wound coil.

12. (canceled)

13. (canceled)

14. (canceled)

15. An inductive coil, comprising:
a semi-circular base;
a semi-circular apex;
a first side connecting a first end of the semi-circular base to a first end of the semi-circular apex; and
a second side connecting a second end of the semi-circular base to a second end of the semi-circular apex,
wherein at least the first side comprises a continuous concave curve.

16. The inductive coil of claim 15, wherein the second side comprises a continuous convex curve.

17. The inductive coil of claim 16, wherein the first side and the second side each have an arc length of at least forty (40) degrees.

18. The inductive coil of claim 15, wherein the inductive coil has a center arc extending from a semi-circular base to a center of the semi-circular apex, and wherein the center arc has a length that is at least two times a radius of the semi-circular base.

19. The inductive coil of claim 15, wherein a radius of the semi-circular apex is less than a radius of the semi-circular base.

20. The inductive coil of claim 15, wherein a radius of the semi-circular apex is the same as a radius of the semi-circular base.

21. The inductive coil of claim 15, wherein the inductive coil is a printed circuit board (PCB) coil.

22. The inductive coil of claim 15, wherein the inductive coil is a wire wound coil.

23. (canceled)

24. (canceled)

25. (canceled)

26. (canceled)

27. An apparatus, comprising:
a housing having a concave curved first edge and a convex curved second edge; and
an inductive coil located within the housing,
wherein the inductive coil has an elongate length with a first elongate side positioned adjacent the concave curved first edge of the housing and a second elongate side positioned adjacent the convex curved second edge of housing, and wherein the first elongate side of the inductive coil has a concave curvature.

28. The apparatus of claim 27, wherein the second elongate side of the inductive coil has a convex curvature.

29. The apparatus of claim 27, wherein the first elongate side and the second elongate side of the inductive coil each have length of at least forty (40) degrees.

30. The apparatus of claim 27, wherein the inductive coil is a printed circuit board (PCB) coil.

31. The apparatus of claim 27, wherein the inductive coil is a wire wound coil.

32. The apparatus of claim 27, wherein the inductive coil comprises an electrical connector configured to electrically connect the inductive coil to a transceiver.

33. The apparatus of claim 27, wherein the inductive coil has a reniform shape.

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. A method, comprising:
positioning a behind-the-ear device on an ear of a recipient, wherein the behind-the-ear device comprises a housing and an external concave coil located within the housing, and wherein an implantable component comprising an implantable coil configured to be implanted in the recipient; and
transcutaneously transferring signals between the behind-the-ear device and the implantable component via the external concave coil and the implantable coil.

53. The method of claim 52, wherein positioning the behind-the-ear device on the ear of the recipient comprises:
positioning the behind-the-ear device on the ear without assistance of a magnet within the housing.

54. The method of claim 52, wherein positioning the behind-the-ear device on the ear of the recipient comprises:
positioning the behind-the-ear device on the ear such that there is an angular offset between the external concave coil and the implantable coil,
wherein the transcutaneously transferring of the signals occurs in the presence of the angular offset.

55. The method of claim 52, wherein transcutaneously transferring signals between the behind-the-ear device and the implantable component via the external concave coil and the implantable coil comprises:
transcutaneously transferring data signals from the behind-the-ear device to the implantable component.

56. The method of claim 52, wherein transcutaneously transferring signals between the behind-the-ear device and the implantable component via the external concave coil and the implantable coil comprises:
transcutaneously transferring power signals from the behind-the-ear device to the implantable component.

57. The method of claim 52, wherein transcutaneously transferring signals between the behind-the-ear device and the implantable component via the external concave coil and the implantable coil comprises:
transcutaneously transferring data signals from the implantable component to the behind-the-ear device.

58. The method of claim 52, wherein the implantable coil is a concave coil.

59. (canceled)
